(19) United States
(12) Patent Application Publication
(10) Pub. No.: US 2022/0305181 A1
(43) Pub. Date: Sep. 29, 2022

(54) SYSTEMS, DEVICES, AND METHODS FOR CONTINUOUS AMBULATORY RENAL REPLACEMENT THERAPY

(71) Applicant: NEPHRIA BIO, INC., Portsmouth, NH (US)

(72) Inventors: Ian G. WELSFORD, Stratham, NH (US); Jesse KIM, Seongnam-si (KR)

(21) Appl. No.: 17/701,286

(22) Filed: Mar. 22, 2022

Related U.S. Application Data
(60) Provisional application No. 63/165,059, filed on Mar. 23, 2021.

(51) Int. Cl. A61M 1/16; A61M 1/36; A61M 1/28

(52) U.S. Cl. CPC A61M 1/1694; A61M 1/3649; A61M 1/28; A61M 2205/3317; A61M 2205/3368; A61M 2205/502

(57) ABSTRACT

Described here are systems, devices, and methods of renal replacement therapy. In some variations, a continuous ambulatory dialysis device may comprise a first fluid conduit configured to receive a fluid from a patient, a second fluid conduit configured to output the fluid to the patient, and an electroosmotic pump configured to pump and filter the fluid. The electroosmotic pump may be coupled between the first fluid conduit and the second fluid conduit. The electroosmotic pump may comprise a first electrode configured to adsorb urea in the fluid, a second electrode, and a porous substrate coupled therebetween.

### CROSS-REFERENCE TO RELATED APPLICATIONS

This application claims the benefit of U.S. Provisional Application No. 63/165,059, filed Mar. 23, 2021, the content of which is hereby incorporated by reference in its entirety.

### TECHNICAL FIELD

Devices, systems, and methods herein relate to renal replacement therapy including, but not limited to, continuous ambulatory renal replacement therapy.

### BACKGROUND

An estimated 750,000 people in the United States suffer from end stage renal disease (ESRD). The conventional standard of care for such patients may include dialysis (e.g., in-center, home-based). Patients on dialysis have an increased mortality and morbidity risk compared to the general population. Conventional dialysis therapy regimes are not as effective as a healthy kidney at removing uremic toxins over a broad molecular weight range and higher molecular weight toxins while also preserving plasma proteins (e.g., albumin) essential for normal function. Therefore, patients on dialysis tend to have higher levels of middle and large molecular solutes in plasma with a concomitant impact on morbidity and mortality.

In-center hemodialysis (HD) is the most common form of dialysis and is typically performed periodically (e.g., a four-hour treatment session three times a week). Relative to a continuously operating healthy kidney, periodic renal therapy may place dialysis patients under additional metabolic stress. For example, dialysis patients typically exhibit a sawtooth-like pattern of plasma pH over the course of a week, with acidification occurring in between dialysis sessions and alkylation occurring immediately after dialysis.

Home-based renal therapy such as peritoneal dialysis (PD) and home-based hemodialysis HD generally have a negative impact on a patient's activities of daily living (ADL) due to the therapy requirements of managing large volumes of fluid and complex durable medical equipment in a home setting. Accordingly, it may be desirable to provide systems, devices, and methods for continuous ambulatory renal replacement therapy.

### SUMMARY

Described here are systems, devices, and methods for renal replacement therapy including ambulatory dialysis. Generally, a continuous ambulatory dialysis device may comprise a first fluid conduit configured to receive a fluid from a patient, a second fluid conduit configured to output the fluid to the patient, and an electroosmotic pump configured to pump and filter the fluid. The electroosmotic pump may be coupled between the first fluid conduit and the second fluid conduit. The electroosmotic pump may comprise a first electrode configured to adsorb urea in the fluid, a second electrode, and a porous substrate coupled therebetween.

In some variations, the first electrode may be configured to adsorb a protein-bound uremic toxin of the urea. In some variations, the protein-bound uremic toxin may comprise one or more of indoxyl sulfate, p-cresyl sulfate, kynurenic acid, and indole-3-acetic acid. In some variations, the first electrode may comprise a porous bilayer polymer. In some of these variations, the first electrode may comprise a sulfonated poly(arylene ether sulfone) polymerized with a metal organic framework linker. In some of these variations, the metal-organic framework linker may comprise one or more of aluminum, iron, and UiO-66. In some of these variations, the first electrode may comprise a sulfonated poly(arylene ether sulfone) polymerized with a polyamide linker. In some of these variations, the first electrode may comprise a sulfonated poly(arylene ether sulfone) polymerized with a MXene linker. In some variations, the porous substrate may comprise an insulator (e.g., dielectric material).

In some variations, the first fluid conduit and the second fluid conduit may each be configured to be coupled to a peritoneal dialysis tubing set. In some variations, the first fluid conduit and the second fluid conduit may each be configured to couple to a peritoneal cavity of the patient. In some variations, a hemodialysis device may be coupled to the second fluid conduit. In some variations, the electroosmotic pump may be configured for continuous dialysis at a rate of up to about 60 mL/hour.

In some variations, a housing may be configured to be worn on a body or a limb of the patient. The housing may comprise the first fluid conduit, the second fluid conduit, and the electroosmotic pump. In some variations, the housing may comprise a disposable component. In some variations, a durable component may comprise a processor and a memory configured to couple to the electroosmotic pump. The durable component may be configured to be releasably coupled to the disposable component.

In some variations, one of the first fluid conduit and the second fluid conduit may comprise an osmolarity sensor configured to generate an osmolarity signal corresponding to the fluid. In some variations, a processor and memory may be coupled to the electroosmotic pump. The processor may be configured to generate a fluid flow rate signal to the electroosmotic pump based on the osmolarity signal. In some variations, a pressure sensor may be configured to generate a pressure signal corresponding to an orthostatic blood pressure.

Also described here are methods comprising pumping a fluid using an electroosmotic pump comprising a porous electrode, and adsorbing a protein-bound uremic toxin of urea in the fluid to the porous electrode of the electroosmotic pump. In some variations, the protein-bound uremic toxin may comprise one or more of indoxyl sulfate, p-cresyl sulfate, kynurenic acid, and indole-3-acetic acid.

In some variations, the electroosmotic pump may be coupled to a body or a limb. In some variations, the electroosmotic pump may be coupled to a peritoneal cavity of the patient. In some variations, the electroosmotic pump may be coupled to a hemodialysis device.

In some variations, pumping may comprise a fluid flow rate of up to about 60 mL/hour. In some variations, an osmolarity of the fluid may be measured, and a fluid flow rate of the fluid may be set based on the measured osmolarity. In some variations, an orthostatic blood pressure of the fluid may be measured.

In some variations, the electrode may comprise a porous bilayer polymer. In some variations, the electrode may comprise a sulfonated poly(arylene ether sulfone) polymerized with a metal organic framework linker. In some variations, the metal-organic framework linker may comprise UiO-66. In some variations, the electrode may comprise a sulfonated poly(arylene ether sulfone) polymerized with a polyamide linker. In some variations, the electrode may comprise a sulfonated poly(arylene ether sulfone) polymerized with a MXene linker.

Also described here are osmolarity sensors comprising a substrate comprising a carbon nanotube, and a first electrode and a second electrode each disposed on the substrate. The first electrode may be interdigitated with the second electrode. In some variations, the first electrode and the second electrode may comprise gold.

### BRIEF DESCRIPTION OF THE DRAWINGS

FIG. 1 is a schematic diagram of an illustrative variation of a peritoneal dialysis system.

FIG. 2 is a schematic diagram of an illustrative variation of a hemodialysis system.

FIG. 3 is a block diagram of an illustrative variation of a renal replacement therapy system.

FIG. 4 is a block diagram of an illustrative variation of a renal replacement therapy system.

FIG. 5A is a perspective view of an illustrative variation of a renal replacement therapy device. FIG. 5B is an exploded perspective view of the device depicted in FIG. 5A.

FIG. 6 is a schematic diagram of an illustrative variation of an electroosmotic pump.

FIGS. 7A-7C are exploded perspective views of illustrative variations of an electroosmotic pump.

FIG. 8 is a set of plots corresponding to an illustrative variation of an electroosmotic pump.

FIG. 9 is a schematic block diagram of an illustrative variation of an electroosmotic pump.

FIG. 10 is a plot of particle concentrations corresponding to use of an electroosmotic pump.

FIG. 11 is a chemical equation of an illustrative variation used in a method of forming a polymer for an electrode.

FIG. 12 is a chemical equation of an illustrative variation used in a method of forming a polymer for an electrode.

FIGS. 13A and 13B are magnified images of an electrode comprising a porous bilayer polymer.

FIG. 14 is an equation of an illustrative variation used in a method of forming a polymer for an electrode.

FIG. 15 are images of an illustrative variation of a polymer for an electrode.

FIG. 16 is a set of plots corresponding to an illustrative variation of electrode adsorption.

FIG. 17 is a plot of MWCO and MWRO for a set of polymers.

FIGS. 18-20 are tables of comparative test results for a set of polymers.

FIG. 21 are top, plan, and cross-sectional side view schematic diagrams of an illustrative variation of an osmolarity sensor.

FIG. 22 is an image of an illustrative variation of an osmolarity sensor.

FIG. 23 is a schematic diagram of an illustrative variation of an osmolarity sensor.

FIG. 24 is a plot of impedance over time of an illustrative variation of an osmolarity sensor.

FIG. 25 is a schematic diagram of an illustrative variation of a peritoneal dialysis process.

FIG. 26 is a schematic diagram of an illustrative variation of a hemodialysis process.

### DETAILED DESCRIPTION

Described here are systems, devices, and methods for renal replacement therapy, such as continuous ambulatory peritoneal dialysis and hemodialysis. As described in more detail herein, a dialysis device may be configured to mimic kidney function. For example, systems and devices may be configured to simultaneously filter and pump fluid using an electroosmotic pump having a compact form factor and weight comfortable enough to be worn by a patient, thereby lowering the impact of renal therapy on a patient's activities of daily living (ADL). In some variations, the electroosmotic pump may function as a filter or include a filter (e.g., membrane) configured to improve waste removal (e.g., urea binding) and which may facilitate recirculation of dialysate, thus reducing dialysate use. Furthermore, the electroosmotic pump may be configured to minimize clotting and pH/salt imbalance due to filtration.

Conventional dialysis generally operates based on convective ultrafiltration (CU) where solutes pass through a set of membrane pores based on a pressure gradient. However, conventional CU does not remove uremic toxins over a broad molecular weight range. For example, conventional dialysis does not remove protein-bound uremic toxins (PBUTs) because the proteins having PBUTs are not captured by (e.g., do not fit) CU membrane pores such that PBUTs (e.g., indoxyl sulfate, p-cresyl sulfate) are returned to the patient and remain in the blood. PBUT leeching from proteins may have negative (e.g., toxic) effects on patients over time if not adequately removed. The systems, devices, and methods as described herein may be configured to remove (e.g., adsorb) uremic toxins over a broad molecular weight range, thus providing higher clearance rates per unit flow rate than conventional dialysis.

In some variations, a renal replacement therapy system may comprise a portable and modular (e.g., cartridge-based) device worn by a patient and configured for ambulatory hemodialysis or peritoneal dialysis for a predetermined duration (e.g., 10 hours per day). In some variations, the renal replacement therapy system may be worn on an arm (e.g., for hemodialysis) or around a waist (e.g., for peritoneal dialysis). In some variations, the system may be compact and portable in size (e.g., having dimensions similar to a smartphone or other mobile device) and weight (e.g., under about 1 lb.). In some variations, the renal replacement therapy system may be configured as a sterile, single-use disposable or as a single patient, durable (e.g., multi-use, reusable, rechargeable) component. Accordingly, the cost and environmental impact of renal therapy may be reduced. In some variations, the renal replacement therapy systems and methods described herein may be used in conjunction with (e.g., supplement, bridge) or supplant conventional dialysis therapy. For example, renal replacement therapy may include slow, low efficiency daily dialysis (SLEDD).

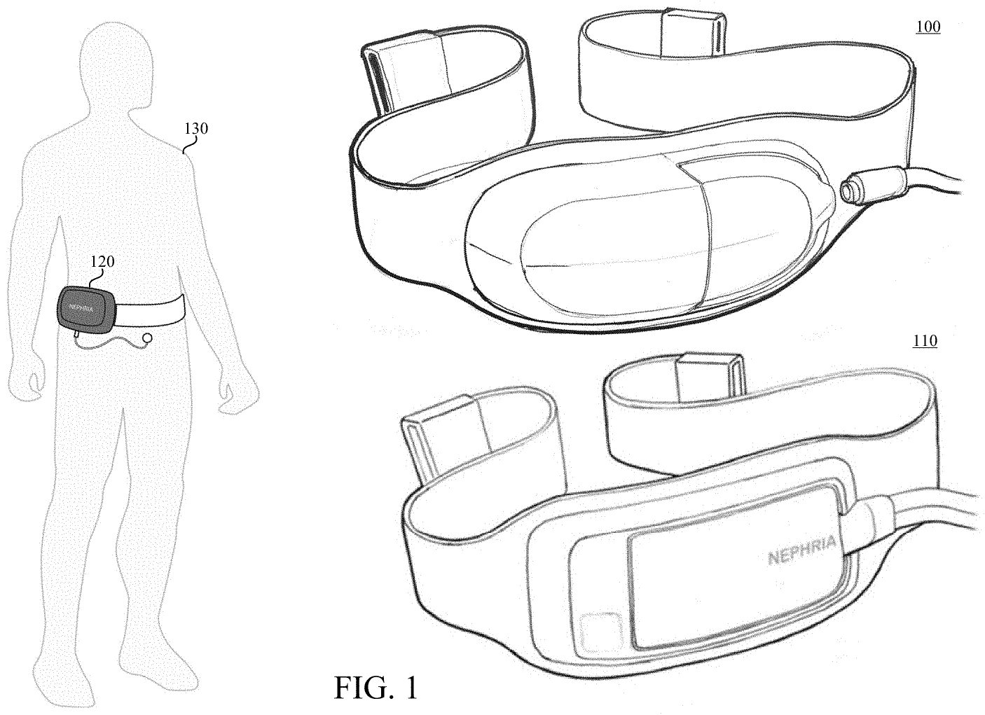

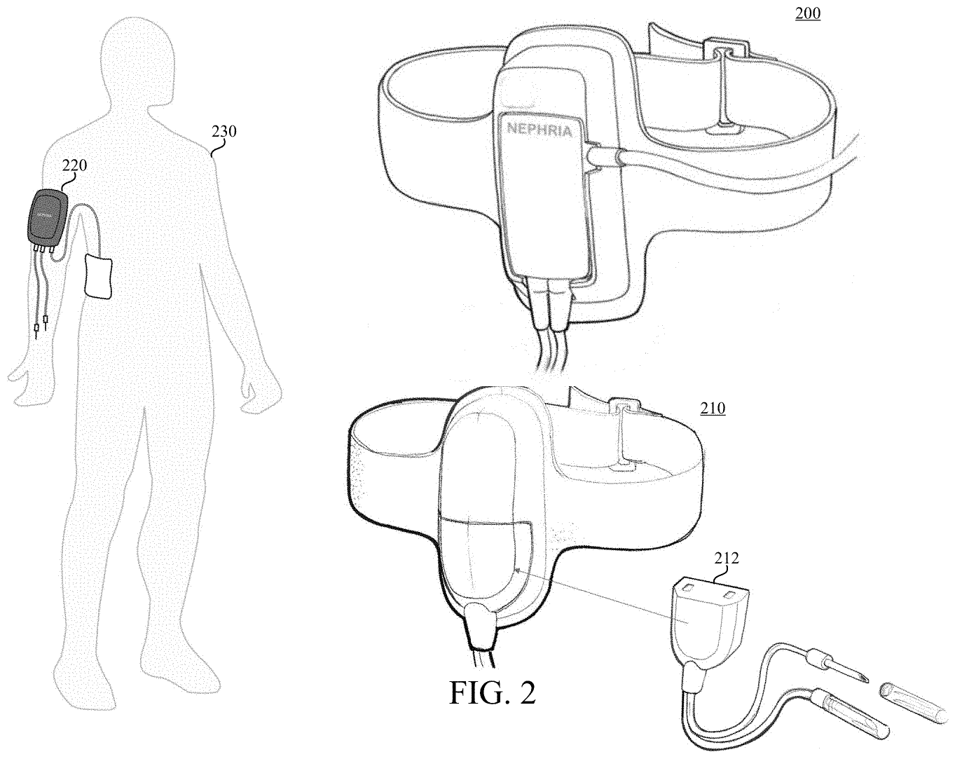

FIG. 1 is a schematic diagram of illustrative variations of continuous ambulatory peritoneal dialysis systems 100, 110, 120 configured to be worn on a body of a patient 130. For example, dialysis may be continuously performed by the systems 100, 110, 120 where dissolved solutes are continuously cleansed from the peritoneal cavity for a predetermined period of time. FIG. 2 is a schematic diagram of illustrative variations of continuous ambulatory hemodialysis systems 200, 210, 220 configured to be worn around a limb (e.g., arm) of a patient 230. In some variations, a hemodialysis system 210 may comprise one or more shunt (e.g., fistula) connectors 212 suitable for patient use in a home setting. Each of the systems 100, 110, 120, 200, 210, 220 may comprise a durable component and a disposable component (e.g., cartridge) as described in more detail herein.

In some variations, a continuous ambulatory dialysis device may comprise a first fluid conduit configured to receive a fluid from a patient, a second fluid conduit configured to output the fluid to the patient, and an electroosmotic pump configured to pump and filter the fluid. The electroosmotic pump may be coupled between the first fluid conduit and the second fluid conduit. The electroosmotic pump may comprise a first electrode configured to adsorb urea in the fluid, a second electrode, and a porous substrate coupled therebetween.

In some variations, a method may comprise pumping a fluid (e.g., dialysate) using an electroosmotic pump comprising a porous electrode, and adsorbing a protein-bound uremic toxin of urea in the fluid to the porous electrode of the electroosmotic pump.

In some variations, an osmolarity sensor may comprise a substrate comprising a carbon nanotube. A first electrode and a second electrode may each be disposed on the substrate. The first electrode may be interdigitated with the second electrode.

### I. Systems and Devices

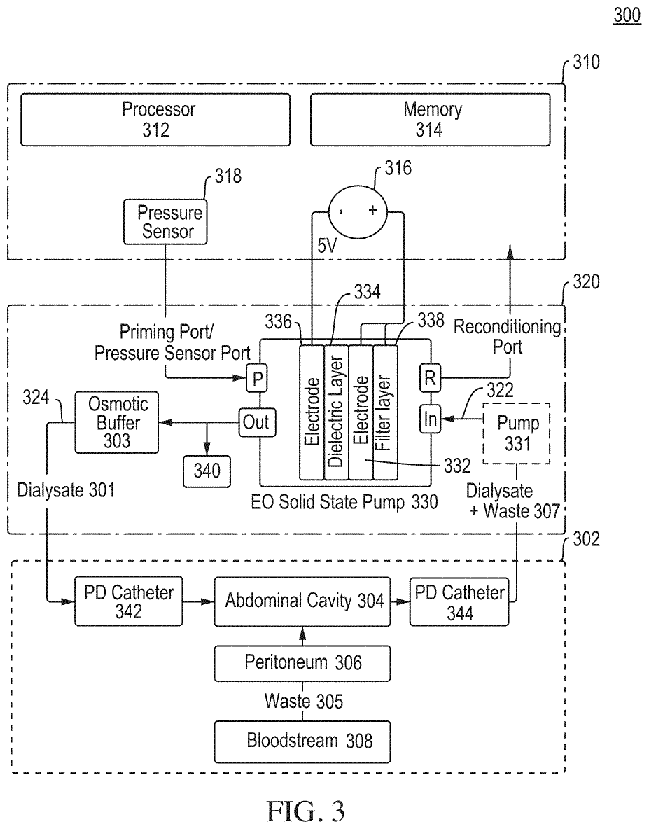

Generally, a renal replacement therapy system may include one or more of the components necessary to treat a patient using the devices as described herein. In some variations, a dialysis device may be configured for one or more of peritoneal dialysis and hemodialysis. FIG. 3 is a block diagram of an illustrative variation of a renal replacement therapy system 300 configured for peritoneal dialysis. For example, the system 300 may comprise a durable component 310 (e.g., housing) and a disposable component 320 (e.g., cartridge) in fluid communication with a patient 302. The durable component 310 may be a reusable portion of the system 300 while the disposable component 310 may be replaced after a predetermined amount of usage (e.g., single use, limited use). The durable component 310 may provide long-term functionality given proper maintenance (e.g., cleaning, charging). The durable component 310 may be releasably coupled to the disposable component 320. The system 300 may be releasably coupled to the patient 302 for performing peritoneal dialysis. For example, the system 300 may be worn on a body of a patient (e.g., FIG. 1).

In some variations, the durable component 310 of the device 300 may comprise one or more of a processor 312, a memory 314, a power source 316, and a pressure sensor 318 (e.g., in-line pressure sensor). Optionally, the durable component 310 may further comprise one or more of an input device, an output device, and a communication device as described in more detail herein. The processor 312 and memory 314 may be configured to control the device 300 including operation of an electroosmotic pump 330 of the disposable component 320. The power source 316 may be configured to power (e.g., DC voltage) the electroosmotic pump 330. The pressure sensor 318 may be configured to measure orthostatic blood pressure.

In some variations, the disposable component 320 may comprise an electroosmotic pump 330 (e.g., electroosmotic solid state pump), an osmotic buffer 303, and an osmolarity sensor 340 (e.g., plasma osmolarity sensor), and an optional fluid pump (e.g., peristaltic pump). The disposable component 320 may be configured to couple to a first catheter 342 (e.g., peritoneal dialysis catheter) which is coupled to an abdominal cavity 304 of the patient 302. Waste products 305 in the bloodstream 308 may be filtered by a peritoneum 306 of the patient 302. Dialysate 301 introduced into the abdominal cavity 304 through the first catheter 342 may receive waste products 305 and a second catheter 344 (e.g., peritoneal dialysis catheter) coupled to the abdominal cavity 304 may receive the dialysate 307 comprising the waste products.

In some variations, the electroosmotic pump 330 may be configured to simultaneously circulate the fluid (e.g., dialysate 301, 307) through the disposable component 320 and the patient 302 and filter out waste products from the fluid (e.g., dialysate 307). In some variations, the electroosmotic pump 330 may comprise a porous first electrode 332, a porous second electrode 336, and a porous substrate 334 (e.g., dielectric layer, membrane) coupled therebetween. In some variations, the electroosmotic pump 330 may be coupled to and controlled by one or more of the durable component 310 (e.g., a processor 312, a memory 314, a power source 316, a pressure sensor 318) and an osmolarity sensor 340. In some variations, the osmolarity sensor 340 may be configured to measure osmotic and saline balance.

In some variations, a filter 338 (e.g., exchange membrane) may be coupled to the first electrode 332. For example, the filter 338 may be coated to the first electrode 332. Additionally or alternatively, the first electrode 332 may comprise the filter 338. That is, the first electrode 332 may be composed of the material of the filter 338.

Additionally or alternatively, the disposable component 320 may comprise an optional pump 331 (e.g., peristaltic pump) coupled (e.g., in fluid communication with) the electroosmotic pump 330.

In some variations, a continuous ambulatory dialysis device 300 may comprise a first fluid conduit 322 configured to receive a fluid 307 from the patient 302, a second fluid conduit 324 configured to output the fluid 301 to the patient 302, and an electroosmotic pump 330 configured to pump and filter the fluid 307. The electroosmotic pump 330 may be coupled between the first fluid conduit 322 and the second fluid conduit 324. The electroosmotic pump 330 may comprise a first electrode 332, 338 configured to adsorb urea in the fluid 307, a second electrode 336, and a porous substrate 334 coupled therebetween.

In some variations, the first fluid conduit 322 and the second fluid conduit 324 may each be configured to be coupled to a peritoneal dialysis tubing set 342, 344. In some variations, the first fluid conduit 322 and the second fluid conduit 324 may each be configured to couple to a peritoneal cavity 304 of the patient 302. In some variations, the electroosmotic pump 330 may be configured for continuous dialysis at a rate of up to about 60 mL/hour.

In some variations, a housing 320 (e.g., disposable component) may be configured to be worn on a body or a limb of the patient. The housing 320 may comprise the first fluid conduit 322, the second fluid conduit 324, and the electroosmotic pump 330. In some variations, a durable component 310 may comprise a processor 312 and a memory 314 configured to couple to the electroosmotic pump 330. The durable component 310 may be configured to be releasably coupled to the disposable component 320.

In some variations, one of the first fluid conduit 322 and the second fluid conduit 324 may comprise an osmolarity sensor 340 configured to generate an osmolarity signal corresponding to the fluid 301, 307. In some variations, the processor 312 may be configured to generate a fluid flow rate signal to the electroosmotic pump 330 based on the osmolarity signal. In some variations, a pressure sensor 318 may be configured to generate a pressure signal corresponding to an orthostatic blood pressure.

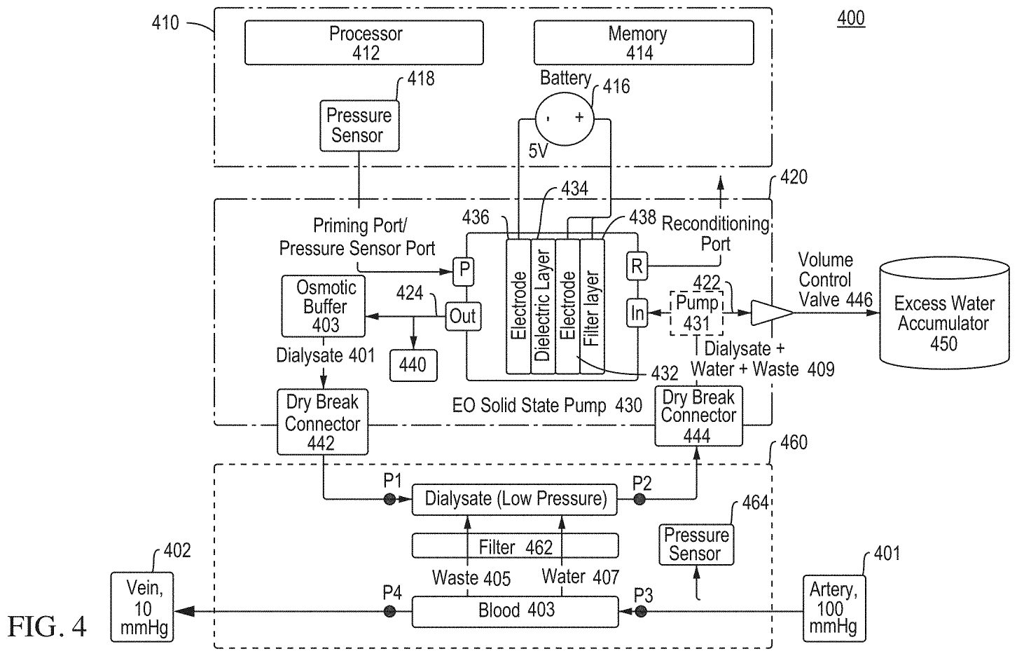

FIG. 4 is a block diagram of an illustrative variation of a renal replacement therapy system 400 configured for hemodialysis. For example, the system 400 may comprise a durable component 410 (e.g., housing) and a disposable component 420 (e.g., cartridge) in fluid communication with a hemodialysis device 460 (e.g., hemodialysis exchange manifold, countercurrent exchange device). The durable component 410 may be a reusable portion of the system 400 while the disposable component 410 may be replaced after a predetermined amount of usage (e.g., single use, limited use). The durable component 410 may provide long-term functionality given proper maintenance (e.g., cleaning, charging). The durable component 410 may be releasably coupled to the disposable component 420. The system 400 may be releasably coupled to the hemodialysis device 460 for performing hemodialysis. For example, the durable component 410 and disposable component 420 may be worn on a body of a patient (e.g., FIG. 2). The hemodialysis device 460 may be releasably coupled to a patient. For example, the hemodialysis device 460 may be configured to receive blood 403 from an artery 401 and return blood 403 to a vein 402. For example, blood flow through the hemodialysis device 460 may be established via an implanted AV fistula where the device 460 receives arterial blood and returns it to a vein continuously during treatment.

In some variations, the durable component 410 of the device 400 may comprise one or more of a processor 412, memory 414, power source 416, and pressure sensor 418 (e.g., in-line pressure sensor). Optionally, the durable component 310 may further comprise one or more of an input device, an output device, and a communication device as described in more detail herein. The processor 412 and memory 414 may be configured to control the device 400 including operation of an electroosmotic pump 430 of the disposable component 420. The power source 416 may be configured to power the electroosmotic pump 430. The pressure sensor 418 may be configured to generate a pressure signal corresponding to an orthostatic blood pressure. In some variations, the valve 446 may be configured to direct fluid to the reservoir 450 based on the pressure signal.

In some variations, the disposable component 420 may comprise an electroosmotic pump 430 (e.g., electroosmotic solid state pump), an osmotic buffer 403, an osmolarity sensor 440 (e.g., plasma osmolarity sensor), a first connector 342, a second connector 444, a valve 446, and an optional fluid pump (e.g., peristaltic pump). The disposable component 420 may be configured to couple the first connector 342 and the second connector 444 (e.g., dry break connectors) to the hemodialysis device 460. Waste products 405 and/or water 407 in blood 405 passing through hemodialysis device 460 may be filtered by a filter 462 into dialysate 401. Dialysate 401 circulated into the hemodialysis device 460 may receive waste products 405 and water 407 for filtering by electroosmotic pump 430. In some variations, the electroosmotic pump 430 may be configured to simultaneously circulate the fluid (e.g., dialysate 401, 409) through the hemodialysis device 460 and filter out waste products from the fluid (e.g., dialysate 409).

In some variations, the electroosmotic pump 430 may comprise a porous first electrode 432, a porous second electrode 436, and a porous substrate 434 (e.g., dielectric layer, membrane) coupled therebetween. In some variations, the electroosmotic pump 430 may be coupled to and controlled by one or more of the durable component 410 (e.g., processor 412, memory 414, power source 416, pressure sensor 418) and osmolarity sensor 440. In some variations, the osmolarity sensor 440 may be configured to measure osmotic and saline balance. In some variations, the osmolarity sensor 340, 440 may comprise an impedance-based carbon nanotube sensor integrated within a fluid conduit (e.g., printed onto a base of a fluid flow path) as described in more detail herein.

In some variations, a filter 438 (e.g., exchange membrane) may be coupled to the first electrode 432. For example, the filter 438 may be coated to the first electrode 432. Additionally or alternatively, the first electrode 432 may comprise the filter 438. That is, the second electrode 436 may be composed of the material of the filter 438, as described in more detail herein. In some variations, the first electrode 432 and the filter 438 of the electroosmotic pump 430 and filter 462 of the hemodialysis device 460 may be composed of one or more of the same materials.

Within the hemodialysis device 460, blood flow is facilitated by arterial blood pressure while the disposable component 420 applies a fluid force against the arterial blood pressure to provide a continuous flow of dialysate 401, 409 to the device 460. Thus, the pressure and flow of dialysate 401, 409 generated by the electroosmotic pump 430 of the disposable component 420 against the blood (e.g., countercurrent flow) facilitates balanced pressure across the filter 462 to reduce hydrostatic water loss. In some variations, the fluid flow rate generated by the electroosmotic pump 430 may be configured to compensate for changes in orthostatic blood pressure.

Additionally or alternatively, the disposable component 320 may comprise an optional pump 431 (e.g., peristaltic pump 1100) coupled (e.g., in fluid communication with) the electroosmotic pump 430.

In some variations, the disposable component 420 may be coupled (e.g., in fluid communication with) to a reservoir 450 (e.g., water accumulator, waste container, waste bag) configured to receive excess fluid from the system 400. For example, a valve 446 may be coupled to a reservoir 450.

In some variations, a continuous ambulatory dialysis device 400 may comprise a first fluid conduit 422 configured to receive a fluid 409 from a patient, a second fluid conduit 424 configured to output the fluid 401 to the patient, and an electroosmotic pump 430 configured to pump and filter the fluid 409. The electroosmotic pump 430 may be coupled between the first fluid conduit 422 and the second fluid conduit 424. The electroosmotic pump 430 may comprise a first electrode 432, 438 configured to adsorb urea in the fluid 409, a second electrode 436, and a porous substrate 434 coupled therebetween.

In some variations, a hemodialysis device 460 may be coupled to the first fluid conduit 422 and the second fluid conduit 424. In some variations, the electroosmotic pump 430 may be configured for continuous dialysis at a rate of up to about 60 mL/hour.

In some variations, a housing 420 (e.g., disposable component) may be configured to be worn on a body or a limb of the patient. The housing 420 may comprise the first fluid conduit 422, the second fluid conduit 424, and the electroosmotic pump 430. In some variations, a durable component 410 may comprise a processor 412 and a memory 414 configured to couple to the electroosmotic pump 430. The durable component 410 may be configured to be releasably coupled to the disposable component 420.

In some variations, one of the first fluid conduit 422 and the second fluid conduit 424 may comprise an osmolarity sensor 440 configured to generate an osmolarity signal corresponding to the fluid 401, 409. In some variations, the processor 412 may be configured to generate a fluid flow rate signal to the electroosmotic pump 430 based on the osmolarity signal. In some variations, a pressure sensor 418 may be configured to generate a pressure signal corresponding to an orthostatic blood pressure.

In some variations, the systems 300, 400 may be configured to filter a fluid (e.g., dialysate) at a fluid flow rate of up to about 1 ml/min for up to about 10 hours on a single battery charge. In some variations, the systems 300, 400 may comprise a user interface (e.g., display, touchscreen, switch, audio feedback, haptic feedback). In some variations, at the completion of a therapy session, patient fluid contacting components of the system 300, 400 (e.g., disposable component 320, 420) may be disposed of and the system 300, 400 (e.g., durable component 310, 410) may be recharged. The systems 300, 400 may be configured to be coupled to (e.g., worn on) a patient similar to the manner shown in FIGS. 1 and 2. In some variations, the systems 300, 400 may have a compact form factor and weight suitable for wearing on a body or limb of a patient. For example, the systems 300, 400 may have a length of about 140 mm and about 160 mm, a width of between about 60 mm and about 70 mm, a height of about 5 mm and about 15 mm, and a weight of between about 0.5 lb and about 1.0 lb, including all ranges and sub-values in-between.

In some variations, the first electrode 432 may be configured to adsorb a protein-bound uremic toxin of the urea. In some variations, the protein-bound uremic toxin may comprise one or more of indoxyl sulfate, p-cresyl sulfate, kynurenic acid, and indole-3-acetic acid. In some variations, the first electrode 432 may comprise a porous bilayer polymer. In some of these variations, the first electrode 432 may comprise a sulfonated poly(arylene ether sulfone) polymerized with a metal organic framework linker. In some of these variations, the metal-organic framework linker may comprise UiO-66. In some of these variations, the first electrode 432 may comprise a sulfonated poly(arylene ether sulfone) polymerized with a polyamide linker. In some of these variations, the first electrode 432 may comprise a sulfonated poly(arylene ether sulfone) polymerized with a MXene linker. In some variations, the porous substrate 434 may comprise an insulator (e.g., dielectric material).

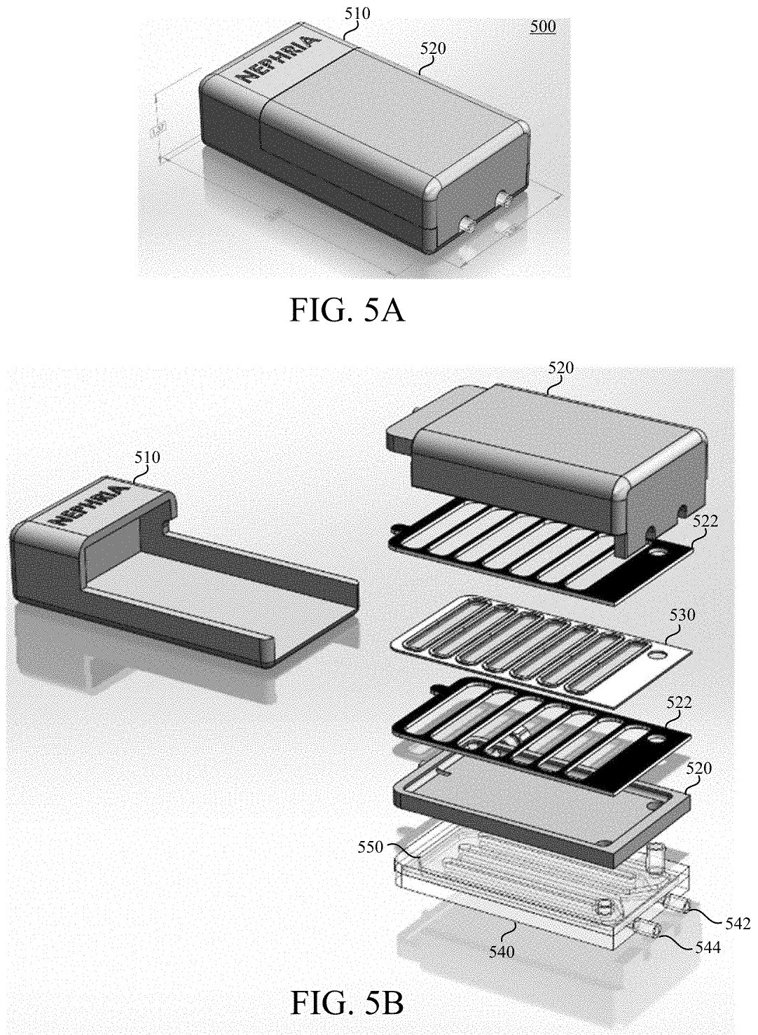

FIG. 5A is a perspective view of an illustrative variation of a renal replacement therapy device 500 comprising a durable component 510 and a disposable component 520 configured to be releasably coupled to the durable component 510. As shown in FIG. 5B, the disposable component 520 may comprise a housing 520 (e.g., chassis, body, substrate, cover) configured to enclose an electroosmotic pump 530 (e.g., electroosmotic pump 330, 430) coupled to a set of fluid seals 522. In some variations, the durable component 510 may optionally further comprise a dialysis device 540 (e.g., hemodialysis exchange manifold) coupled in fluid communication with the electroosmotic pump 530 in a manner similar to as shown and described with respect to FIG. 4. The dialysis device 540 may comprise a filter 550 (e.g., filter 462) configured to remove waste and water from blood, an input 542 configured to receive arterial blood, and an output 544 configured to output blood to a vein.

A. Electroosmotic Pump

Generally, the electroosmotic pumps described herein may be configured to circulate a fluid such as dialysate while also filtering out waste products from the fluid (e.g., urea). For example, applying a voltage to a pair of porous electrodes having a porous substrate coupled therebetween results in an electrochemical reaction where positive ions produced by an anode move with the fluid towards a cathode, thereby generating fluid pressure and pumping fluid through the pump.

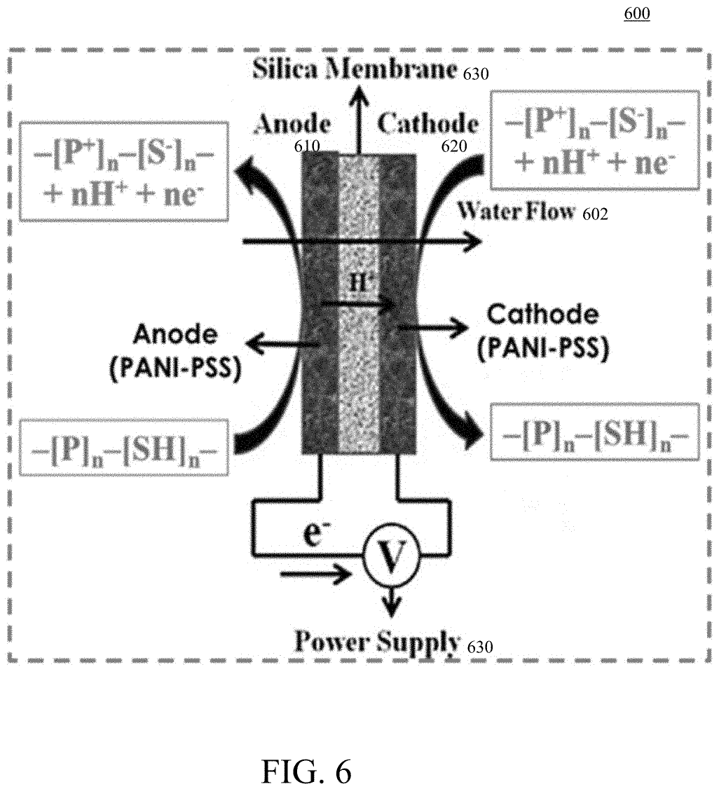

FIG. 6 is a schematic diagram of an illustrative variation of an electroosmotic pump 600. An electroosmotic pump 600 may comprise a solid state electrode configured to facilitate fluid flow through porous (e.g., permeable) electrodes 610, 620 and a porous substrate 630 (e.g., membrane, silica frit) via electroosmosis. In some variations, fluid 602 may flow from an anode 610 to a cathode 620. For example, the electrodes 610, 620 may comprise a conductive carbon. For example, the porous electrodes 610, 620 may comprise carbon (e.g., carbon paper, carbon woven fabric) and an electrochemical reaction material (e.g., silver (Ag)/silver oxide (AgO), MnO(OH), polyaniline, etc.). The electroosmotic pump 600 may be coupled to a power source 630 (e.g., power supply).

In some variations, the electroosmotic pumps herein may comprise one or more electroosmotic pumps described in U.S. Pat. No. 11,015,583, issued on May 25, 2021, the contents of which are hereby incorporated by reference in its entirety.

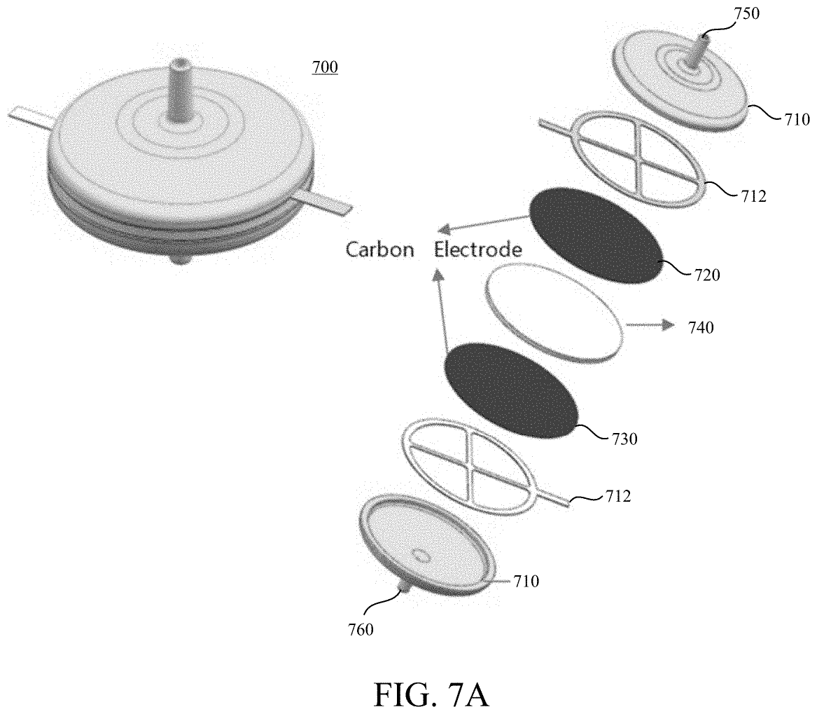

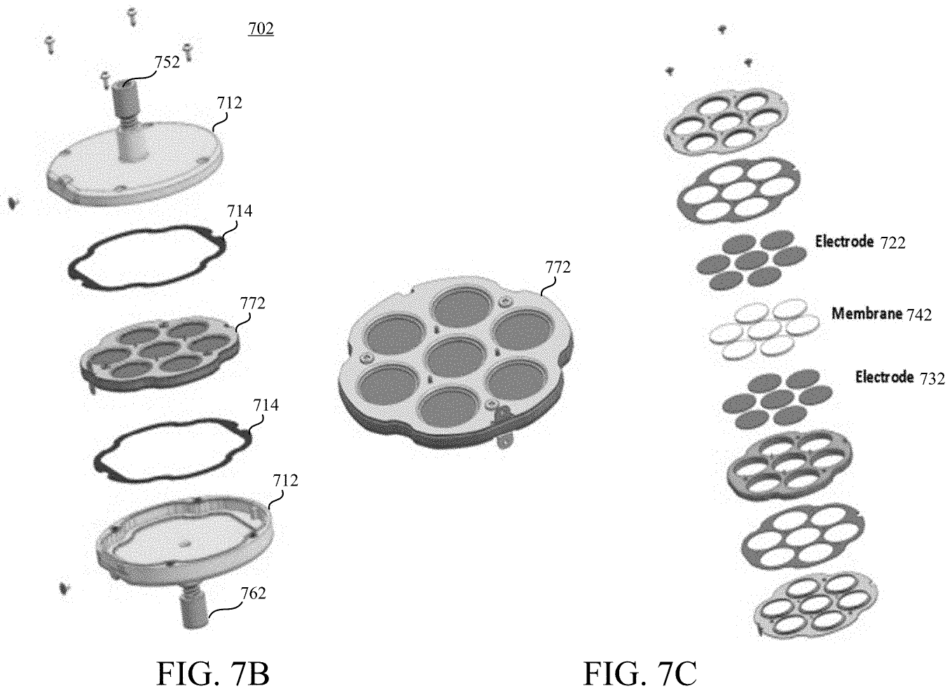

FIGS. 7A-7C are exploded perspective and exploded views of illustrative variations of an electroosmotic pump 700. FIG. 7A is an exploded perspective view of an electroosmotic pump 700 comprising a housing 710, a set of fluid seals 712, a first electrode 720, a second electrode 730, and a porous substrate 740 coupled between the first electrode 720 and the second electrode 730. The housing 710 may comprise an inlet 750 and an outlet 760 configured to couple to corresponding fluid conduits. In some variations, a surface area of the electroosmotic pump 700 may be between about 5 cm² and about 30 cm², including all ranges and sub-values in-between.

FIG. 7B is an exploded perspective view of an electroosmotic pump 702 comprising a housing 712, a set of fluid seals 714, an electrode assembly 772, an inlet 752, and an outlet 762. FIG. 7C is an exploded perspective view of the electrode assembly 772 comprising a first electrode 722, a second electrode 732, and a porous substrate 742 (e.g., membrane) coupled between the first electrode 722 and the second electrode 732. In some variations, the porous substrate 742 may comprise silica and comprise a pore radius of about 300 nm. In some variations, a surface area of the electroosmotic pump 702 may be between about 290 cm² and about 300 cm², including all ranges and sub-values in-between. In some variations, the electroosmotic pump 702 may be configured to generate a fluid flow rate of about 0.7 mL/min and provided at a maximum pressure of about 160 kPa (about 23 psi) at 5 V.

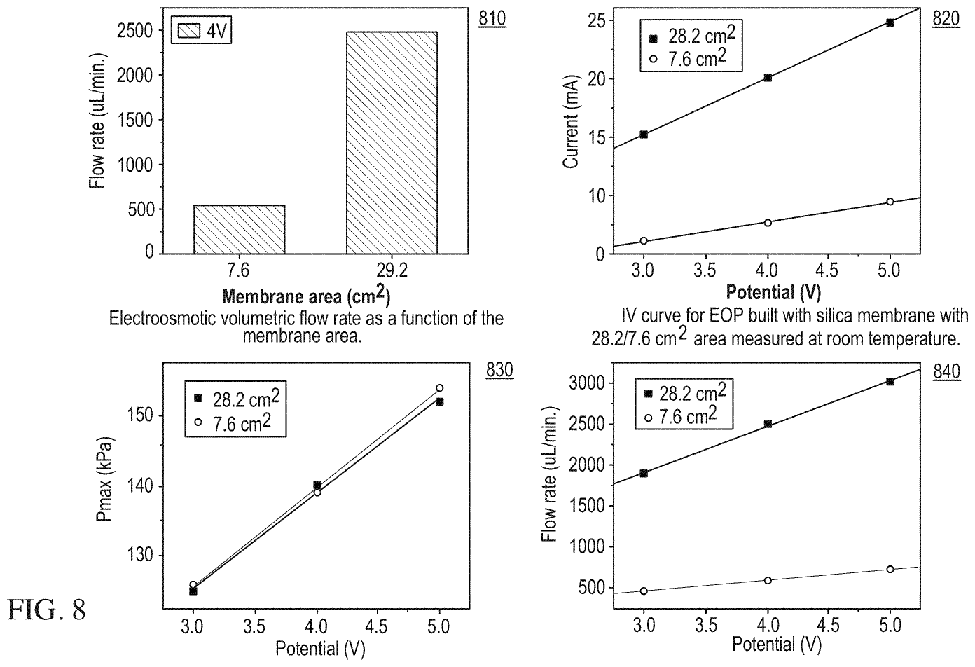

FIG. 8 is a set of plots corresponding to an illustrative variation of an electroosmotic pump. Plot 810 corresponds to an electroosmotic volumetric flow rate as a function of surface area of the pump. Plot 820 corresponds to current as a function of voltage. Plot 830 corresponds to pressure as a function of voltage. Plot 840 corresponds to a volumetric flow rate as a function of voltage. In some variations, fluid flow generated by an electroosmotic pump as described herein may scale generally linearly with voltage and area.

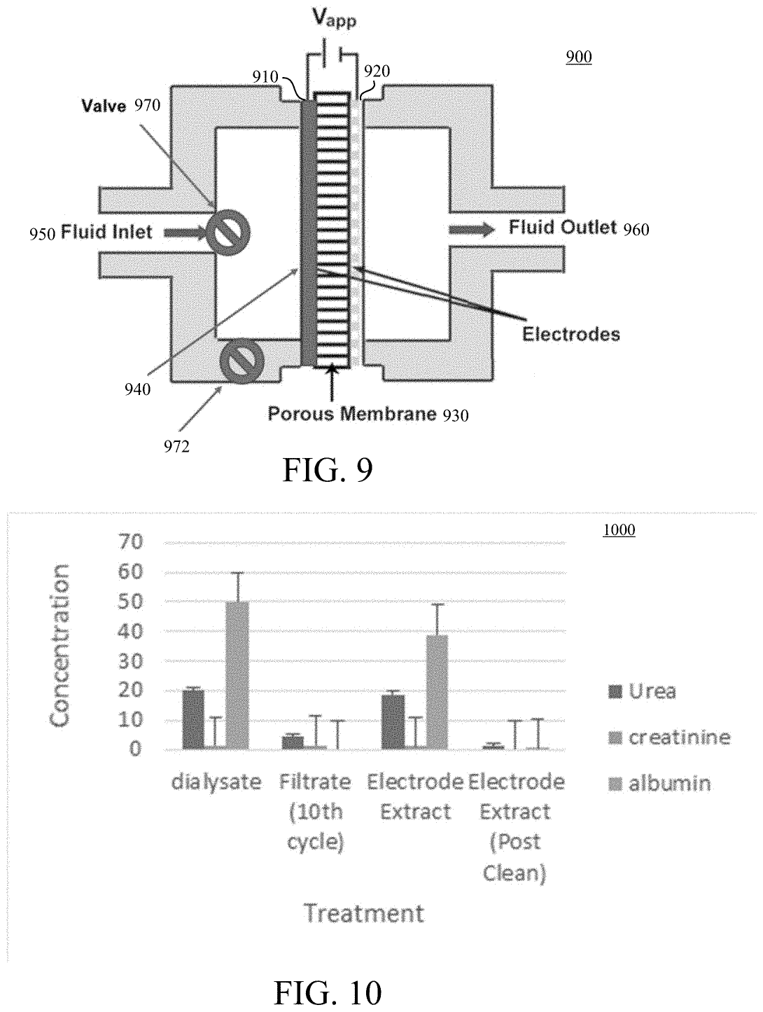

FIG. 9 is a schematic block diagram of an illustrative variation of an electroosmotic pump 900 coupled between a first fluid conduit 950 (e.g., fluid inlet) and a second fluid inlet 960 (e.g., fluid outlet). Fluid received through the first fluid conduit 950 may be configured to flow through the electroosmotic pump 900. The electroosmotic pump 900 may comprise a first electrode 910 comprising a filter 940, a second electrode 920, and a porous substrate 930 (e.g., porous membrane, frit) coupled therebetween. A voltage applied to the electroosmotic pump 900 may pump and filter the fluid. For example, the filter 940 may adsorb waste products (e.g., urea) from the fluid. The first electrode 910 may be configured as an anode and the second electrode 920 may be configured as a cathode. In some variations, the first electrode 910 may itself be the filter 940 or a filter 940 may be coupled to a surface of the first electrode 910. For example, the filter 940 may be coated to a surface of the first electrode 910.

In some variations, the electrodes 910, 920 may comprise a set of pores having a pore size of between about 0.1 μm and about 500 μm, between about 5 μm and about 300 μm, and between about 10 μm and about 200 μm, including all ranges and sub-values in-between. In some variations, the electrodes 910, 920 may comprise a porosity of between about 5% and about 95%, between about 50% and about 90%, and between about 60% and about 80%, including all ranges and sub-values in-between.

In some variations, the porous substrate 930 may comprise an insulator such as one or more of spherical silica, porous silica, porous alumina, rockwool, gypsum, ceramic, cement, polymer resin, rubber, urethane, glass, natural fiber, combinations thereof, and the like. Polymer resin may include, but is not limited to, a synthetic fiber such as polypropylene, polyethylene terephthalate, polyacrylonitrile. A natural fiber may include, but is not limited to, wool, cotton, and a sponge. Glass may include, but is not limited to glass wool, glass frit, and porous glass.

In some variations, the porous substrate 930 may comprise a thickness of between about 20 μm and about 10 mm, between about 300 μm and about 5 mm, and between about 1000 μm and about 4 mm, including all ranges and sub-values in-between. In some variations, spherical silica may comprise a diameter of between about 20 nm and about 500 nm, between about 30 nm and about 300 nm, between about 40 nm and about 200 nm, including all ranges and sub-values in-between.

FIG. 10 is a plot 1000 of particle concentrations corresponding to use of an electroosmotic pump. Dialysate comprised an initial concentration of urea of about 20 mg/dL, an initial concentration of creatinine of about 1.2 mg/dL, and an initial concentration of albumin was about 50 g/dL. A filtrate was sampled after electroosmotic pump filtration, and an electrode extract of captured solutes was sampled after 10 cycles. A post clean of the electrode extract was performed after polarity switching across the electrodes of the electroosmotic pump to release the solute, thereby facilitating reuse of the electroosmotic pump. Moreover, the electroosmotic pumps configured to adsorb protein-bound uremic toxins generated a flow rate of on average of about 112.71% relative to electroosmotic pumps without a filter (e.g., treated electrode).

B. Electrode

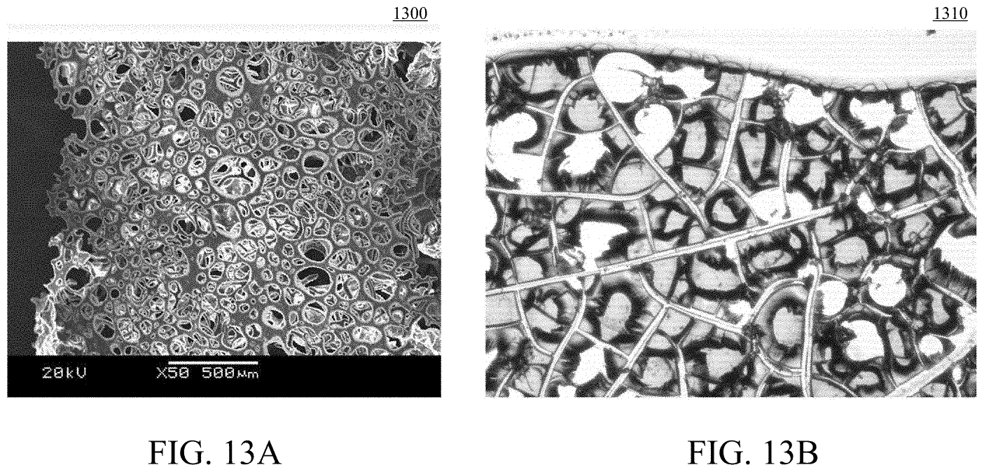

Generally, the electrodes described herein may be configured to adsorb urea in the fluid such as a protein-bound uremic toxin. In some variations, the protein-bound uremic toxin may comprise one or more of indoxyl sulfate, p-cresyl sulfate, kynurenic acid, indole-3-acetic acid, and the like. In some variations, the electrode may comprise a sulfonated poly(arylene ether sulfone) polymerized with an alkyl linker such as a metal organic framework linker (e.g., UiO-66), a polyamide linker, and a MXene linker. The alkyl linker may be polymerized between adjacent subunits of polymers (e.g., SPAES) and functions as a solute binding unit (SBU) where the solute is a protein-bound uremeic toxin. The alkyl linker facilitates bilayer formation in the polymer having a set of pores for filtration and an adsorption layer (e.g., alkyl cage) for capture of protein-bound uremic toxins. For example, FIGS. 13A and 13B are magnified images of an electrode 1300, 1310 comprising a porous bilayer polymer. The electrodes described herein may exhibit improved biocompatibility, sieving coefficient, and urea binding affinity over conventional electrodes.

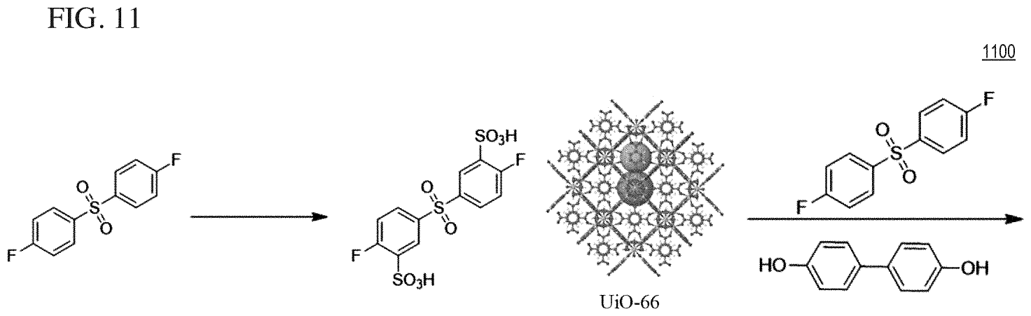

FIG. 11 depicts a chemical equation 1100 of an illustrative variation of a method of forming a polymer for an electrode where a polymerization reaction occurs due to an alkyl linker (e.g., metal organic framework linker, UiO-66) between subunits to form a SPAES polymer.

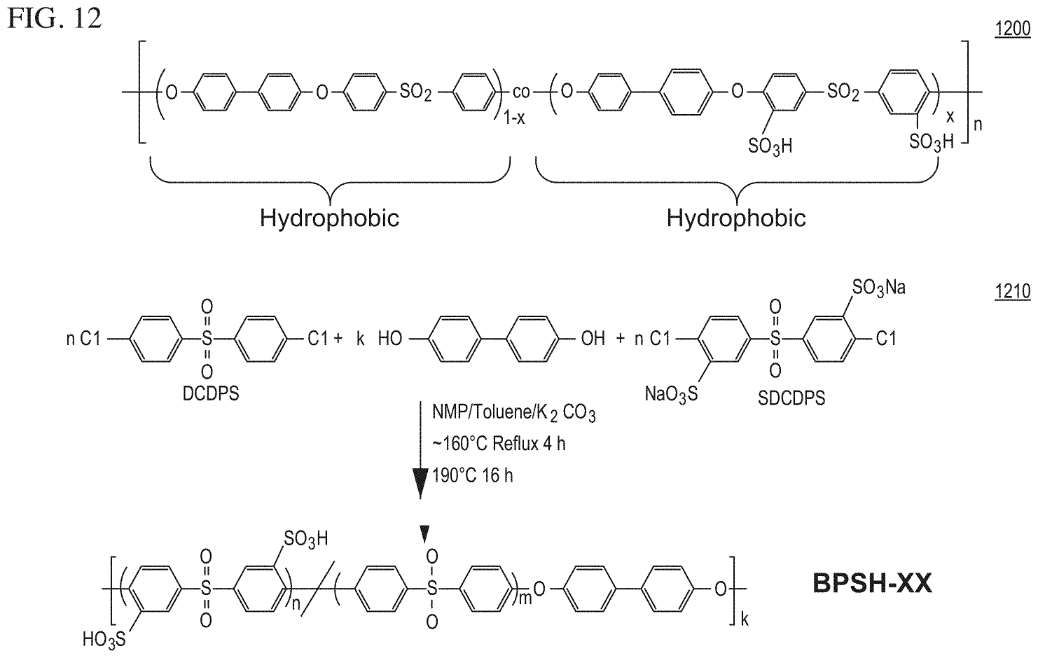

FIG. 12 is a chemical equation 1200 of an illustrative variation of a method of direct polymerization of pre-sulfonated monomers. A method of forming the copolymers 1210 may include a nucleophilic aromatic substitution step polymerization of 4,4′-dichlorodiphenyl sulfone biphenol and a predetermined amount of the sulfonated analog (e.g., SDCPS).

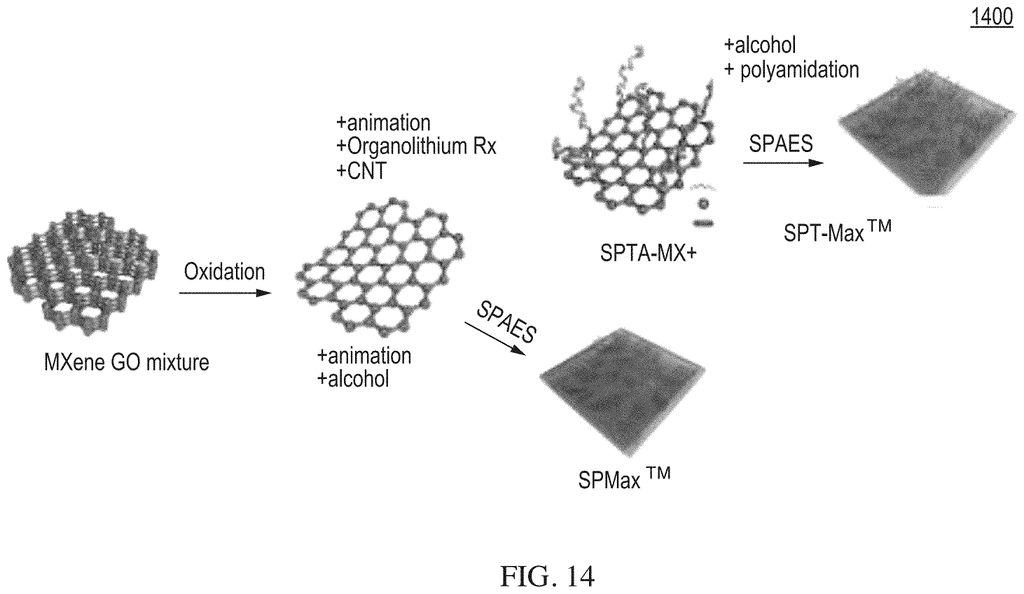

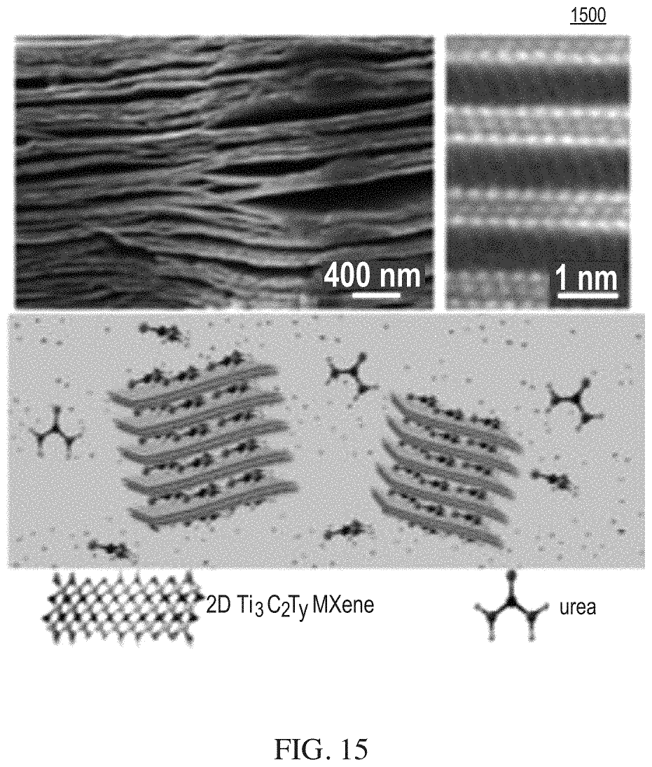

FIG. 14 is a flowchart 1400 of an illustrative variation of a method of forming a polymer for an electrode. In some variations, Ti₃C₂ MXenes may be complexed with graphene oxide and polyethylarylsulfone (PAES) using a combination of oxidation, alcohol treatment and amination to form a SP-Max polymer that combines the advantages of PAES and MXenes. To minimize pH disruption and coagulation, PAES may be combined with both SPTA and a carbon nanotube coating through organolithium reactions, amination, and polyamidation treatment to make a SPT-Max polymer. FIG. 15 are images of an illustrative variation of a MXene polymer of an electrode including Ti₃C₂ MXenes.

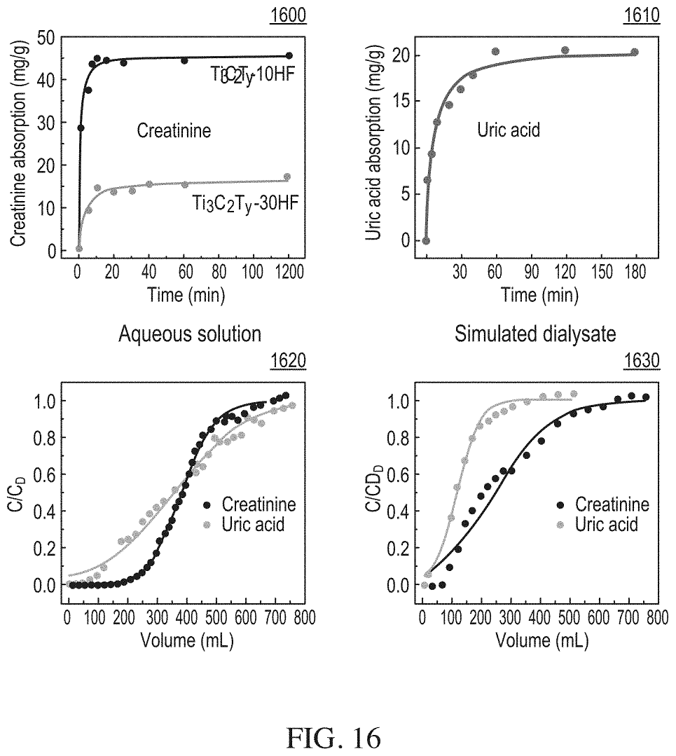

FIG. 16 is a set of plots corresponding to an illustrative variation of electrode adsorption. For example, plot 1600 corresponds to creatinine adsorption as a function of time. Plot 1610 corresponds to uric acid adsorption as a function of time. Plot 1620 corresponds to a concentration of creatinine and uric acid as a function of volume for an aqueous solution. Plot 1630 corresponds to a concentration of creatinine and uric acid as a function of volume for a dialysate.

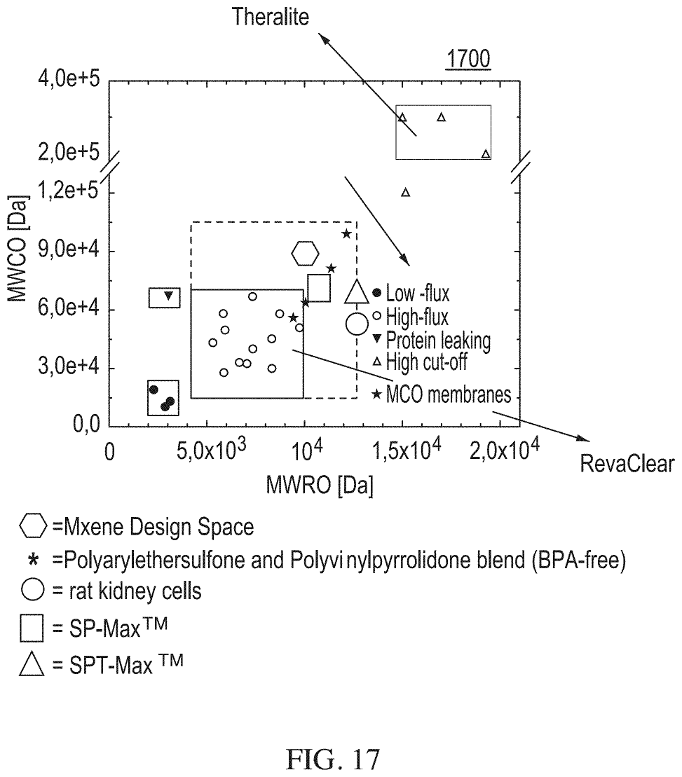

FIG. 17 is a plot 1700 of MWCO and MWRO for a set of polymers and rat kidney cells. Higher permeability membrane polymers may facilitate higher efficiency (e.g., shorter duration) dialysis treatment sessions. However, conventional synthetic membrane polymers have limitations associated with molecular weight cut off (MWCO) and permeability while maintaining acceptable levels of biocompatibility. The black downward arrow denotes change in slope to approach idealized natural kidney function. Plotting the results of the SP-Max and SPT-Max membrane polymers compared to other membranes as well as published results for rat kidney, the SP membrane polymer performs favorably to native kidney performance. As shown in FIG. 1, the SP-Max (SP-Max and SPT-Max) membrane polymers more closely approximate the MWCO/MWRO ratios seen in rat kidneys, a finding which supports their use in long-term hemodialysis.

A sieving profile of SP-Max and SPT-Max polymer was determined both dry and prewetted for 1 hour. The effective pore size (Stokes-Einstein radius) was estimated from filtration experiments before and after dialysate exposure, and results were compared to hydrodynamic radii of middle and large uremic toxins and essential proteins. The ability of the polymers to remove large uremic toxins while ensuring the retention of albumin was directly compared to the Revaclear® 500 and the Theralite® cartridges. Urea removal was calculated using an artificial dialysate containing 3.5 g/dL albumin, 50 mmol/L urea, and 3 mg/dL creatinine. Dialysate was infused through cartridges under controlled pressure and temperature (37° C.±2° C.). A UF-Coefficient (mL/(h*mmHg) was measured using bovine blood, Hct 32%, Pct 60 g/L, 37° C. This value was divided by m of effective area of a membrane polymer to get (mL/(h*mmHg)/m). KoA urea was calculated at QB=300 mL/min, QD=500 mL/min, UF=0 mL/min. Sieving coefficients were measured with bovine plasma, QB=300 mL/min, UF=60 mL/min. Clearances In-Vitro was measured at UF=0 mL/min±10%. Measurements were taken at Kₜ/Vᵤᵣₑₐ values>1.5. The sieving coefficient (SC) was calculated according to equation (1) as follows:

$$SC = \frac{2 * {C}_{F}}{{C}_{P} + {C}_{R}} \qquad ( 1 )$$

C_{F} is the concentration of the solute in the filtrate, C_{P} is the concentration in the permeate, and C_{R} is the concentration in the retentate. Filtration experiments were carried out under a constant shear rate (γ=750 s⁻¹) and with an ultrafiltration rate set at 20% of the blood side entrance flux Q_{Bin}, calculated as:

$${Q}_{Bin} = \frac{g * n * p * {d}_{i}^{3}}{32}$$

Q_{Bin} is the flux at the blood side entrance in ml/min, and n is the number of fibers in the cartridge.

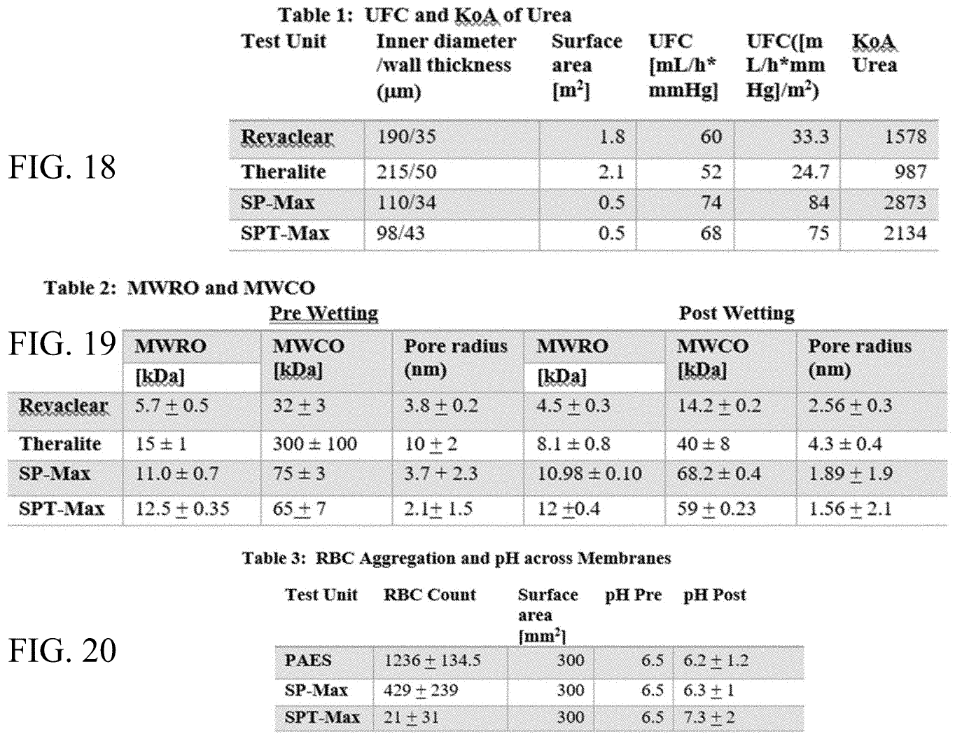

The obtained sieving curves were characterized by their molecular weight retention onset (MWRO) and molecular weight cut-off (MWCO). The MWCO may correspond to the molecular weight at which the sieving coefficient is 0.1. The MWRO may correspond to the molecular weight at which the sieving coefficient is 0.96. Since membrane pore sizes are not discrete but a distribution, the pore size distribution may be described either as the effective pore size (from the MWCO) or the mean pore size from the log-normal distribution. Pore sizes correspond to molar mass where a is the pore radius in Å and MM is a=0.33 (MM)^{0.46}, and the dextran molar mass is in g/mol. The Stokes-Einstein radius at the MWCO may correspond to the effective membrane pore radius. The molecular weight cut-off may be the molecular weight from which at least about 90% of the molecules are retained by the membrane polymer. Therefore, the hydrodynamic radius of that molecule may represent the size of molecules that are retained (at least about 90%), which may be an effective pore. Pore sizes are also described by the Log-normal pore size distribution as mean pore size. Sieving curves were transformed into pore size distributions based the mentioned correlation. The distributions were evaluated as log-normal distributions and characterized by its mean and variance. The performance of membrane polymers versus conventional solutions in terms of UFC is presented in FIG. 18 (e.g., Table 1). UFC/m² was up to about 2.5 times greater than comparators while KoA was 35% greater than the nearest comparator.

FIG. 19 (e.g., Table 2) compares the MWRO and MWCO of each polymer type tested prior to and after wetting. FIG. 20 (e.g., Table 3) lists pore sizes for a set of polymers. The ideal pore radius for kidney filtration (e.g., toxin removal and protein sparing) may average between about 5 and about 10 in the wetted configuration, although this does not take into account the formation of a protein layer at the exchange surface of membrane polymers which may mitigate protein loss. When compared with average estimations of serum components such as albumin (3.5 nm), (32 microglobin (1.7 nm) and Tumor Necrosis Factor (1.9-2.3 nm) which should ideally be retained, the cutoff of the membrane polymers is within the range required for retention of critical factors while removing larger uremic toxins.

The duration of use over which each sorbent material (N=5) maintained at least 90% of its performance was tested (UFC). Theralite® took 5.3±2.5 hours to drop below 90% while Revaclear® took 6.1±3.3 to drop below 90%. In contrast, SP-Max membrane polymers lasted 18±8.7 h before dropping below the 90% threshold, thereby supporting a 10 hour safe use in the devices described herein.

In contrast to SP-Max, SPT-Max may comprise carbon nanotubes (CNT) in a membrane polymer. Carbon nanotube complexation has been shown to decrease coagulation on polymeric membranes used in dialysis and was tested for SPT-Max, SP-Max and untreated SPAES membrane polymers. As shown in Table 3, SPT-Max showed significantly less blood cell aggregation than either uncoated SPAES or SP-Max such that additional processing of the membrane seen in SPT-Max formulation may facilitate diminished heparin pumping in the devices described herein.

Moreover, the ability of the SPT-Max membrane polymer to mitigate some pH alterations during filtration was examined. In N=10 trials, the SPT-Max membrane polymer showed an increase in post flux artificial dialysate versus the starting dialysate which was titrated to a pH of 6.5.

C. Osmolarity Sensor

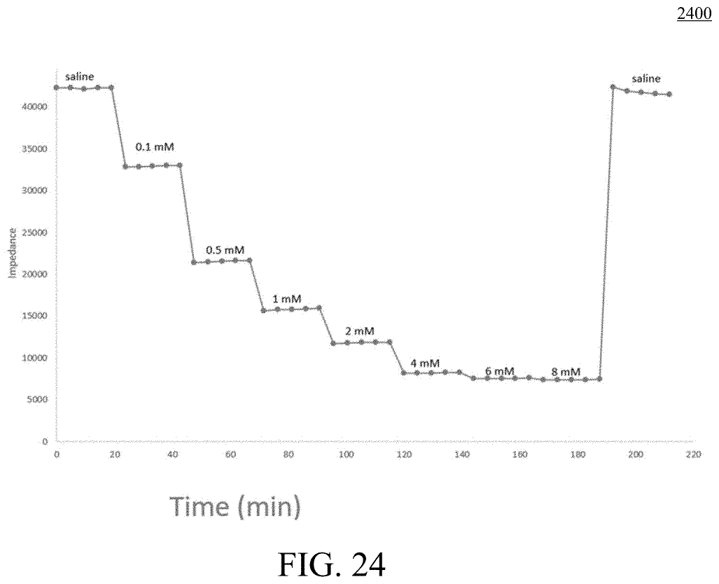

Generally, the osmolarity sensors described herein may be configured to generate an osmolarity signal corresponding to a concentration of a fluid (e.g., dialysate). The osmolarity signal may be used to ensure osmotic and dialysate balance within predetermined thresholds. In some variations, an osmolarity sensor may comprise a carbon nanotube impedance-based sensor configured to measure changes in the osmolarity of blood during dialysis. FIG. 24 is a plot 2400 of impedance over time of an illustrative variation of an osmolarity sensor showing a change in NaCL concentration.

FIG. 21 depicts schematic top, plan, and cross-sectional side view diagrams of an illustrative variation of an osmolarity sensor 2100 comprising a base 2130 (e.g., non-conductive layer, SiO₂) and a non-conductive layer 2131. For example, the base 2130 may have a thickness of about 200 microns. A substrate 2110 comprising a carbon nanotube may be disposed on the non-conductive layer 2130. A first and second electrode 2120, 2122 may be disposed on the substrate 2110. For example, the first and second electrode 2120, 2122 may have a thickness of about 100 nm and a width of about 75 μm. The first electrode 2120 may be interdigitated with the second electrode 2122. For example, a distance (e.g., gap) between the first electrode 2120 and second electrode 2122 may be about 75 μm. A gap between a substrate 2110 of a first electrode 2120 and a substrate 2110 of a second electrode 2122 may be about 35 μm. In some variations, the osmolarity sensor 2100 may have a length of about 2 mm and a width of about 1 mm.

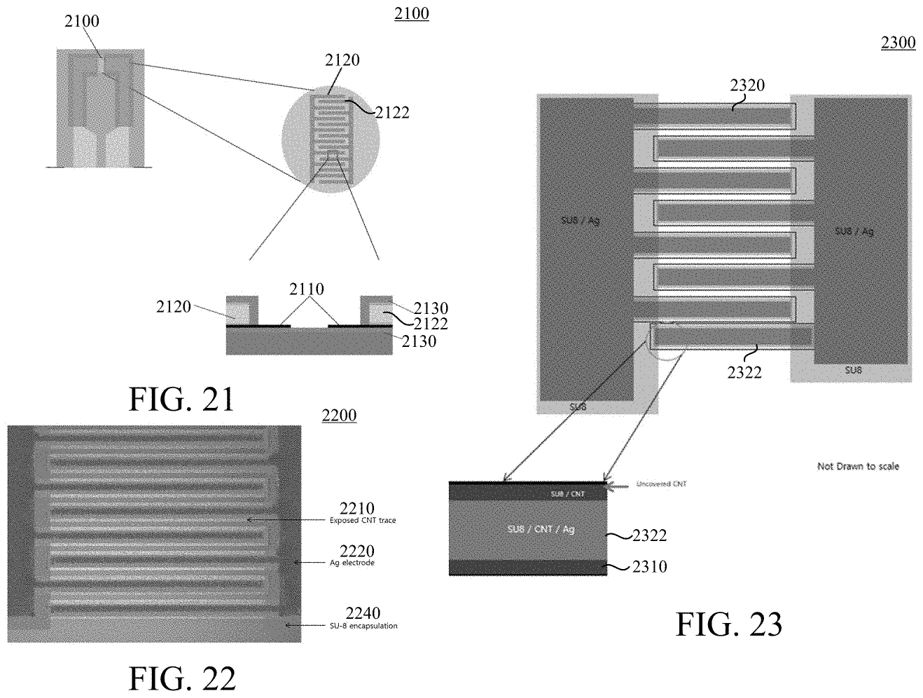

FIG. 22 is an image of an illustrative variation of an osmolarity sensor 2200 including a carbon nanotube substrate 2210, a electrode 2220 (e.g., gold electrode), and a base 2240 (e.g., SU-8 encapsulation). In some variations, the sensor may be printed on a polymer matrix of a cartridge with electrical pins only on the impedance circuitry on a PCBA within the durable component of the device.

FIG. 23 is a plan and cross-sectional side view schematic diagram of an illustrative variation of an osmolarity sensor 2300 including a carbon nanotube substrate 2310, a first electrode 2320, and a second electrode 2322.

D. Input Device

Generally, an input device of a dialysis system may serve as a control interface for a patient. In some variations, the system may comprise one or more input devices. For example, the device may comprise an input device configured to control the dialysis device. Additionally or alternatively, a compute device may comprise a corresponding input device (e.g., touchscreen interface) configured to control the dialysis device. In some variations, the input device may be configured to receive input to control one or more of the electroosmotic pump 330, 430, output device, communication device, and the like. For example, patient actuation of an input device (e.g., switch) may be processed by processor 312, 412 and memory 314, 414 to output a control signal to electroosmotic pump 330, 430.

In some variations, an input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

E. Output Device

Generally, the output devices described herein may comprise a graphical user interface configured to permit a patient to view information and/or control a dialysis device. In some variations, a display may comprise at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

In some variations, the dialysis device may comprise an output device such as an audio device and/or haptic device. For example, an audio device may audibly output patient data, fluid data, dialysis data, system data, alarms and/or notifications. For example, the audio device may output an audible alarm when a drain line blockage is detected. In some variations, an audio device may comprise at least one of a speaker, piezoelectric audio device, magnetostrictive speaker, and/or digital speaker. In some variations, a patient may communicate with other users using the audio device and a communication channel. For example, a user may form an audio communication channel (e.g., cellular call, VoIP call) with a remote provider.

In some variations, a haptic device may be incorporated into the dialysis device to provide additional sensory output (e.g., force feedback) to the patient. For example, a haptic device may generate a tactile response (e.g., vibration) to confirm user input to an input device (e.g., touch surface).

F. Processor

A dialysis device 300, 400, as depicted in FIGS. 3 and 4, may comprise a processor 312, 412 and a machine-readable memory 314, 414 in communication with one or more compute devices (not shown). The processor 312, 412 may be connected to the compute devices by wired or wireless communication channels. The processor 312, 412 may be configured to control one or more components of the device 300, 400 such as the electroosmotic pump 330, 430. The processor 312, 412 may be implemented consistent with numerous general purpose or special purpose computing systems or configurations. Various exemplary computing systems, environments, and/or configurations that may be suitable for use with the systems and devices disclosed herein may include, but are not limited to software or other components within or embodied on personal computing devices, network appliances, servers or server computing devices such as routing/connectivity components, portable (e.g., hand-held) or laptop devices, multiprocessor systems, microprocessor-based systems, and distributed computing networks.

The processor 312, 412 may incorporate data received from memory 314, 414 and patient input to control the device 300, 400. The memory 314, 414 may further store instructions to cause the processor 312 to execute modules, processes, and/or functions associated with the device 300, 400. The processor 312, 412 may be any suitable processing device configured to run and/or execute a set of instructions or code and may comprise one or more microcontrollers, data processors, image processors, graphics processing units, physics processing units, digital signal processors, and/or central processing units. The processor 312, 412 may be, for example, a general purpose processor, a Field Programmable Gate Array (FPGA), an Application Specific Integrated Circuit (ASIC), configured to execute application processes and/or other modules, processes, and/or functions associated with the system and/or a network associated therewith. The underlying device technologies may be provided in a variety of component types such as metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, combinations thereof, and the like.

G. Memory

Some variations of memory 314, 414 described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as air or a cable). The media and computer code (also may be referred to as code or algorithm) may be those designed and constructed for a specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical discs; solid state storage devices such as a solid state drive (SSD) and a solid state hybrid drive (SSHD); carrier wave signal processing modules; and hardware devices that are specially configured to store and execute program code such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

H. Communication Device

Generally, the dialysis devices described herein may communicate with networks and computer systems through a communication device. In some variations, the dialysis device 300, 400 may be in communication with other devices (e.g., compute devices) via one or more wired and/or wireless networks. A wireless network may refer to any type of digital network that is not connected by cables of any kind. Examples of wireless communication in a wireless network include, but are not limited to Bluetooth, cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system. In some variations, communication using the communication device may be encrypted.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication. In some variations, a network interface may comprise a radiofrequency receiver, transmitter, and/or optical (e.g., infrared) receiver and transmitter.

I. Power Source

Generally, the dialysis devices described herein may receive power from an internal power source (e.g., battery) and be recharged using an external power source (e.g., wireless charger, wall outlet, base station). The dialysis device may receive power via a wired connection, and/or a wireless connection (e.g., induction, RF coupling, etc.).

### II. Methods

Also described here are methods for dialysis using the devices and systems described herein. Generally, the methods described here comprise pumping fluid (e.g., dialysate) and adsorbing PBUTs from the fluid using an electroosmotic pump. The methods may thus enable high efficiency removal of urea at a lower flow rate than through conventional dialysis.

Generally, a method may comprise pumping a fluid using an electroosmotic pump comprising a porous electrode, and adsorbing a protein-bound uremic toxin of urea in the fluid to the porous electrode of the electroosmotic pump. In some variations, the protein-bound uremic toxin may comprise one or more of indoxyl sulfate, p-cresyl sulfate, kynurenic acid, and indole-3-acetic acid. In some variations, the electroosmotic pump may be coupled to a body or a limb. For example, the electroosmotic pump may be coupled to a peritoneal cavity of the patient or a hemodialysis device.

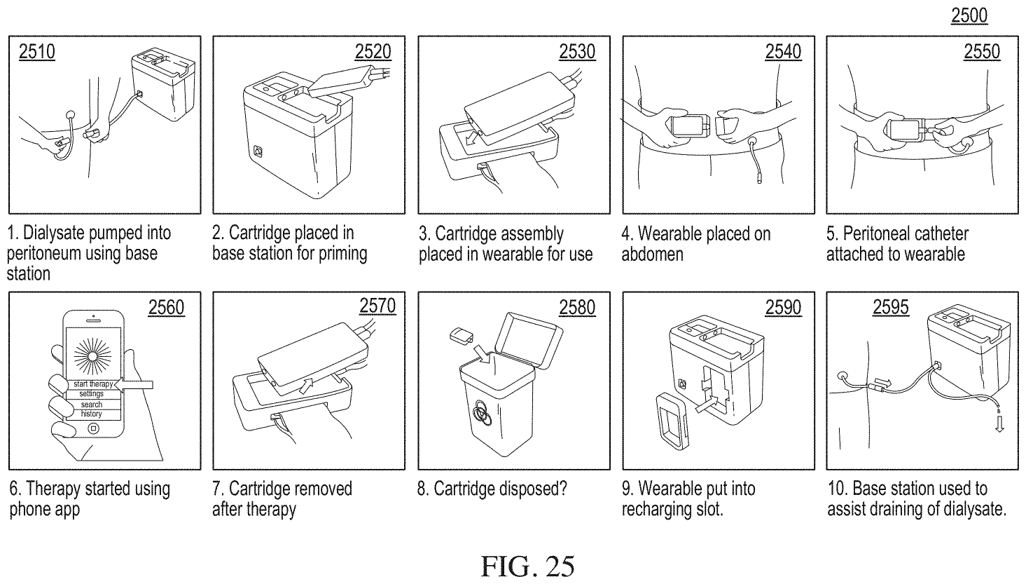

FIG. 25 is a schematic diagram of an illustrative variation of a peritoneal dialysis process 2500. The method 2500 may optionally comprise introducing dialysate into a peritoneum 2510. Optionally, a durable component may be charged and/or primed with sterile dialysate 2520. For example, a power source 316 (e.g., battery) of a durable component 310 may be electrically coupled to a power source of a base station to recharge the power source 316. In some variations, the power source 316 may be charged by a wired connection and/or wirelessly. In some variations, the durable component may be coupled to a disposable component 2530. For example, the disposable component 320, 520 may be placed within a durable component 310, 510. In some variations, the assembled dialysis device may be coupled to the patient 2540. For example, the dialysis device may be worn around a body (e.g., waist) or limb (e.g., arm, leg). In some variations, a catheter may be coupled to the disposable component of the dialysis device 2550. For example, a peritoneal catheter 344 may be connected to an inlet fluid conduit of the disposable component 320. In some variations, a dialysis treatment may be initiated for a predetermined amount of time 2560. For example, a patient may generate a treatment start signal using an input device (e.g., compute device, durable component).

In some variations, the systems and devices described herein may be configured to provide continuous dialysis at a rate of about 60 mL/hour using an electroosmotic pump having a uremic toxin exchange efficiency of up to about 2.5 times that of conventional dialysis sorbent cartridges such that the effective impact is closer to about 150 mL/hour in terms of filtration efficiency. In some variations, a generally lower dialysis flow rate may reduce cardiovascular morbidity and mortality in dialysis patients.

Optionally, an osmolarity of the fluid and an orthostatic blood pressure may be measured, and a fluid flow rate of the fluid may be based on one or more of the measured osmolarity and orthostatic blood pressure.

In some variations, after dialysis treatment has been completed, the dialysis device may be disconnected from the peritoneal catheter and the dialysis device may be disassembled 2570. For example, disposable component 320, 520 may be released from the durable component 310, 510. In some variations, the disposable component may be disposed 2580. Optionally, a durable component may be recharged 2590. Optionally, dialysate may be drained from the patient 2595.

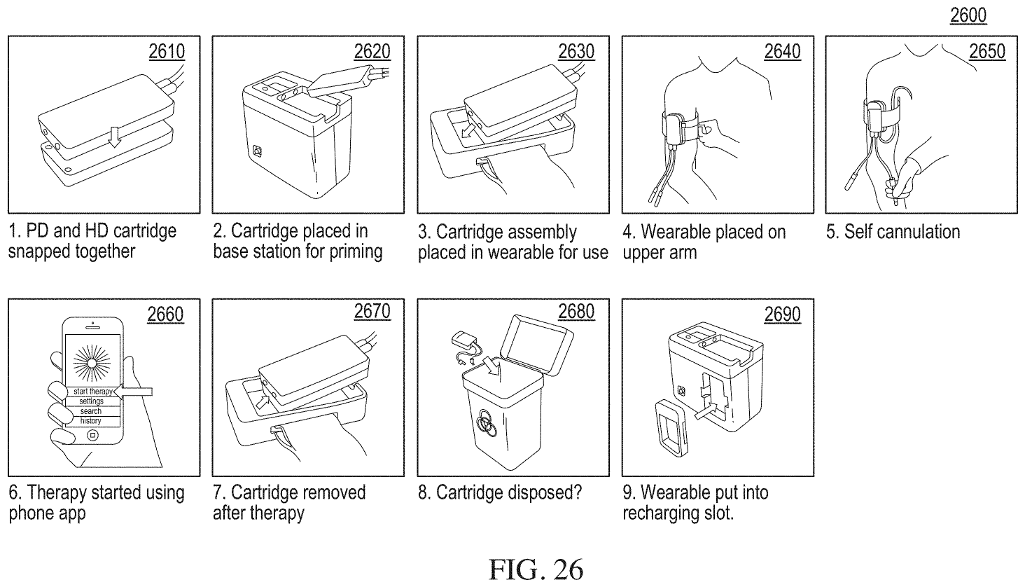

FIG. 26 is a schematic diagram of an illustrative variation of a hemodialysis process 2600. The method 2600 may comprise optionally coupling a disposable component to a hemodialysis device 2610. For example, a disposable component 420, 520 may be coupled to a hemodialysis device 460, 550. Optionally, a durable component may be charged and/or primed with sterile dialysate 2620. For example, a power source 416 (e.g., battery) of a durable component 410 may be electrically coupled to a power source of a base station to recharge the power source 416. In some variations, the power source 416 may be charged by a wired connection and/or wirelessly. In some variations, the durable component may be coupled to a disposable component 2630. For example, the disposable component 420, 520 may be placed within a durable component 410, 510. In some variations, the assembled dialysis device may be coupled to the patient 2640. For example, the dialysis device may be worn around a body (e.g., waist) or limb (e.g., arm, leg). In some variations, a catheter coupled to the disposable component of the dialysis device may be coupled to an artery and vein of a patient 2650. In some variations, a dialysis treatment may be initiated for a predetermined amount of time 2660. For example, a patient may generate a treatment start signal using an input device (e.g., compute device, durable component).

In some variations, the systems and devices described herein may be configured to provide continuous dialysis at a rate of about 60 mL/hour. Furthermore, since conventional high flux HD requires a high flow AV fistula and fistulas tend to decrease in patency over time, the lower flow requirements of the systems and devices described herein may increase the patency of a fistula, thereby increasing the time available for effective therapy for end-stage renal dialysis patients. Although a typical patent AV fistula may have a flow rate of up to about 600 mL/min, cardiovascular risk factors for heart failure due to ventricular hypertrophy may increase as a function of dialysis flow rate such that cardiovascular morbidity and mortality may decrease with lower dialysis flow rates.

Optionally, an osmolarity of the fluid and an orthostatic blood pressure may be measured, and a fluid flow rate of the fluid may be based on one or more of the measured osmolarity and orthostatic blood pressure.

In some variations, after dialysis treatment has been completed, the dialysis device may be disconnected from the peritoneal catheter and the dialysis device may be disassembled 2670. For example, disposable component 320, 520 may be released from the durable component 310, 510. In some variations, the disposable component may be disposed 2680. Optionally, a durable component may be recharged 2690.

Although the foregoing variations have, for the purposes of clarity and understanding, been described in some detail by illustration and example, it will be apparent that certain changes and modifications may be practiced, and are intended to fall within the scope of the appended claims. Additionally, it should be understood that the components and characteristics of the systems and devices described herein may be used in any combination. The description of certain elements or characteristics with respect to a specific figure are not intended to be limiting or nor should they be interpreted to suggest that the element cannot be used in combination with any of the other described elements. For all of the variations described herein, the steps of the methods may not be performed sequentially. Some steps are optional such that every step of the methods may not be performed.

1. A continuous ambulatory dialysis device, comprising:
a first fluid conduit configured to receive a fluid from a patient;
a second fluid conduit configured to output the fluid to the patient; and
an electroosmotic pump configured to pump and filter the fluid, the electroosmotic pump coupled between the first fluid conduit and the second fluid conduit, and the electroosmotic pump comprising a first electrode configured to adsorb urea in the fluid, a second electrode, and a porous substrate coupled therebetween.

2. The device of claim 1, wherein the first electrode is configured to adsorb a protein-bound uremic toxin of the urea.

3. The device of claim 2, wherein the protein-bound uremic toxin comprises one or more of indoxyl sulfate, p-cresyl sulfate, kynurenic acid, and indole-3-acetic acid.

4. The device of claim 1, wherein the first electrode comprises a porous bilayer polymer.

5. The device of claim 4, wherein the first electrode comprises a sulfonated poly(arylene ether sulfone) polymerized with a metal organic framework linker.

6. (canceled)

7. The device of claim 4, wherein the first electrode comprises a sulfonated poly(arylene ether sulfone) polymerized with a polyamide linker.

8.-11. (canceled)

12. The device of claim 1, further comprising a hemodialysis device coupled to the first fluid conduit and the second fluid conduit.

13. The device of claim 1, wherein the electroosmotic pump is configured for continuous dialysis at a rate of up to about 60 mL/hour.

14.-17. (canceled)

18. The device of claim 1, further comprising a processor and memory coupled to the electroosmotic pump, the processor configured to generate a fluid flow rate signal to the electroosmotic pump based on an osmolarity signal.

19. (canceled)

20. A method, comprising:
pumping a fluid using an electroosmotic pump comprising a porous electrode; and
adsorbing a protein-bound uremic toxin of urea in the fluid to the porous electrode of the electroosmotic pump.

21. The method of claim 20, wherein the protein-bound uremic toxin comprises one or more of indoxyl sulfate, p-cresyl sulfate, kynurenic acid, and indole-3-acetic acid.

22. The method of claim 20, further comprising coupling the electroosmotic pump to a body or a limb.

23. The method of claim 20, further comprising coupling the electroosmotic pump to a peritoneal cavity of the patient.

24. The method of claim 20, further comprising coupling the electroosmotic pump to a hemodialysis device.

25. The method of claim 20, wherein pumping comprises a fluid flow rate of up to about 60 mL/hour.

26. The method of claim 20, further comprising measuring an osmolarity of the fluid, and setting a fluid flow rate of the fluid based on the measured osmolarity.

27. The method of claim 20, further comprising measuring an orthostatic blood pressure of the fluid.

28. The method of claim 20, wherein the electrode comprises a porous bilayer polymer.

29. The method of claim 28, wherein the electrode comprises a sulfonated poly(arylene ether sulfone) polymerized with a metal organic framework linker.

30. (canceled)

31. The method of claim 28, wherein the electrode comprises a sulfonated poly(arylene ether sulfone) polymerized with a polyamide linker.

32.-34. (canceled)
